(19) United States
(12) Patent Application Publication
(10) Pub. No.: US 2025/0054636 A1
(43) Pub. Date: Feb. 13, 2025

(54) METHOD FOR OPTIMIZING CHART REVIEW AND AUDIT WORKFLOWS IN ACCURATE RISK ADJUSTMENT AND METHOD THEREOF

(71) Applicant: RAAPID, INC, Louisville, KY (US)

(72) Inventors: Durai Vaithilingam RAMACHANDIRAN, Thanjavur (IN); Pinal PATEL, Mahesana (IN); Raxitkumar GOSWAMI, Tragad (IN)

(73) Assignee: RAAPID, INC, Louisville, KY (US)

(21) Appl. No.: 18/797,409

(22) Filed: Aug. 7, 2024

Related U.S. Application Data
(60) Provisional application No. 63/531,330, filed on Aug. 8, 2023.

(51) Int. Cl. G16H 50/30; G06F 40/279; G06V 30/19; G16H 15/00

(52) U.S. Cl. CPC G16H 50/30; G06F 40/279; G06V 30/19; G16H 15/00

(57) ABSTRACT

The present invention relates to a workflow for chart review and audit of natural language medical text. More specifically, the present invention uses Knowledge Graph to find the ICD-10-CM Codes and MEAT (Monitor, Evaluate, Assess and Treat) evidence for a given diagnosis. The present workflow also provided for feedback collection and consolidation. The present system, instead of processing OCR for all pages, pre-processes to identify the pages that need OCR (i.e., which are the pages containing non-machine readable text—either in full page or partial page—are determined) and passed to the OCR engine. This approach reduces the time, cost and errors associated with the OCR process.

### FIELD OF INVENTION

The present invention relates to a method for the interpretation of patient medical charts. More specifically, the present invention relates to a workflow leveraging advanced technologies such as Natural Language Processing (NLP) and machine learning algorithms. The present invention enables the extraction of structured information from unstructured patient medical charts. The present invention further relates to a novel ICD-10-CM code validation algorithm, integrated with a comprehensive knowledge graph which ensures the accuracy of assigned codes. The invention also introduces the concept of MEAT (Monitor, Evaluate, Assess, and Treat) knowledge, derived from extensive analysis of medical evidence. By incorporating a feedback loop mechanism to continuously improve system performance, the invention aims to improve risk adjustment workflows, optimizing risk assessment and ultimately improving healthcare outcomes.

### BACKGROUND OF THE INVENTION

In the current era, the world has rapidly accepted digitization and the said global migration towards digitization has put focus on the collection and interpretation of data as one of the most critical arenas of innovation. As with other industries, medical practices are characterized by an increasingly overwhelming amount of information, new knowledge in diagnostics and therapeutics, and highly fragmented care environments, with potentially hundreds of different individuals delivering care to a single patient across healthcare settings.

Further, the care and treatment provided to the patients solely depends on the prescription provided by the doctor. Typically, the test reports and prescriptions are provided on the medical charts which may be complex and indecipherable for every individual who is taking care of the patient. Also, the medical chart may include things like a person's medical history, allergies, and other similar facets which are required to be taken care of. Wrong interpretation of the medical chart may harm the patient and disturb the treatment process. In a few instances patients need urgent attention having no time to go to an expert for medical assistance, in such cases there is a need for a system which is adaptable to provide instant cure suggestion by reading medical charts possessing medical data of the patient.

As of now, the interpretation of the data contained in a medical chart of a patient is done by medical professionals manually reading the said medical chart. This method requires a lot of time and expertise in the medical field. Also, manual depiction of the medical chart may lead to a lot of human errors such as inaccurate interpretation of data which may further lead to unwanted financial implications posing grave risks.

However, there are no such solutions available in the arena of healthcare to optimize chart review and chart audit workflows in risk adjustment.

In view of the challenges associated with the above state of the art, there is a need for the introduction of a more streamlined workflow which involves reduced human intervention. The reduced human intervention ensures reduced human error and thus greater accuracy in the workflows along with shorter timelines, ultimately benefiting healthcare organizations and improving patient outcomes.

### Objectives of the Invention

The primary objective of the present invention is to provide a method for interpretation of medical charts.

Another objective of the present invention is to provide a system for implementing the method for interpretation of medical charts.

Another objective of the present invention is to provide the system and method for leveraging advanced technologies such as Natural Language Processing (NLP) and machine learning algorithms in interpretation of the medical charts.

Yet another objective of the present invention is to provide the system and method which is adaptable to extract structured information from the unstructured medical chart.

Yet another objective of the present invention is to provide the system and method which eliminates human intervention for medical chart reading.

Yet another objective of the present invention is to provide the system and method which employed MEAT (Monitor, Evaluate, Assess, and Treat) knowledge graph designed to optimize clinical decision-making and patient care.

Yet another objective of the present invention is to provide the system and method which utilizes the ICD-10-CM codes to group and identify diseases, disorders, symptoms, poisonings, adverse effects of drugs and chemicals, injuries.

Yet another objective of the present invention is the system and method which is adaptable to receive feedback and update the system accordingly in order to improve the system performance.

### SUMMARY OF THE INVENTION

The primary object of the present invention is to provide an innovative workflow to improve the efficiency and accuracy of chart review and chart audit workflows in risk adjustment processes. By leveraging advanced technical components and algorithms, the present invention aims to streamline the extraction of relevant information from unstructured patient medical charts and transform it into structured data.

Another key aspect of the present invention is the implementation of a Natural Language Processing (NLP) pipeline, which effectively extracts critical data from the unstructured text in medical charts. The said NLP pipeline utilizes sophisticated algorithms to identify and extract key elements such as diagnoses, symptoms, and treatments, enabling the conversion of unstructured information into structured, actionable data.

Yet another aspect of the present invention is to introduce an ICD-10-CM code validation algorithm integrated with a knowledge graph. The present invention leverages the extensive medical knowledge contained within the knowledge graph, the algorithm ensures the accuracy and validity of the assigned ICD-10-CM codes, reducing the risk of coding errors and enhancing the overall precision of the chart review and audit processes.

Another significant aspect of this invention is to create and utilize a MEAT (Monitor, Evaluate, Assess, and Treat) knowledge. The said MEAT knowledge is generated based on comprehensive analysis and understanding of the medical evidence, clinical guidelines, and best practices. The said knowledge is employed to identify MEAT evidences within the extracted data, further enhancing the accuracy and reliability of the risk adjustment workflows.

Furthermore, another aspect of the present invention is to incorporate a feedback loop mechanism, facilitated by learning algorithms, to continuously collect user feedback and improve the present system's performance over time. The present workflow iteratively incorporates user input and refines the algorithms, the invention ensures ongoing enhancements in the chart review and audit workflows, leading to increased efficiency and effectiveness in risk adjustment processes.

An important aspect of the present invention is that the present system, instead of processing Optical Character Recognition (OCR) for all pages, pre-processes to identify the pages that need OCR (i.e., which are the pages containing non-machine readable text—either in full page or partial page—are determined) and passed to the OCR engine. This approach reduces the time, cost and errors associated with the OCR process.

### BRIEF DESCRIPTION OF DRAWING

The drawings constitute a part of this invention and include exemplary embodiments of the present invention illustrating various objects and features thereof.

FIG. 1: A simplified block diagram of a detailed level technical workflow and exemplary embodiment of Data Processing System and Method for Accurate Risk Adjustment: Chart Review and Audit of Natural Language Medical Text.

FIG. 2: Illustrates and demonstrates the traversal capabilities of a sample Knowledge Graph, allowing exploration from both the inside out and the outside in. This interactive navigation enhances the understanding of entity interconnections, making our Knowledge Graphs valuable tools for various applications.

FIG. 3: Illustrates the unstructured data from a sample medical chart.

FIG. 4: illustrates an example of a MEAT knowledge graph. In the said example, the system receives the text extracted from an unstructured medical chart.

FIG. 5: illustrates an output, produces by the system, presenting the findings in a structured and meaningful manner.

### DETAILED DESCRIPTION OF THE INVENTION

The following description describes various features and functions of the disclosed system with reference to the accompanying figures. In the figures, similar symbols identify similar components, unless context dictates otherwise. The illustrative aspects described herein are not meant to be limiting. It may be readily understood that certain aspects of the disclosed system can be arranged and combined in a wide variety of different configurations, all of which have not been contemplated herein.

Accordingly, those of ordinary skill in the art will recognize that various changes and modifications of the embodiments described herein can be made without departing from the scope of invention. In addition, descriptions of well-known functions and constructions are omitted for clarity and conciseness.

Features that are described and/or illustrated with respect to one embodiment may be used in the same way or in a similar way in one or more other embodiments and/or in combination with or instead of the features of the other embodiments.

The terms and words used in the following description are not limited to the bibliographical meanings, but, are merely used to enable a clear and consistent understanding of the invention. Accordingly, it should be apparent to those skilled in the art that the following description of exemplary embodiments of the present invention are provided for illustrative purposes only and not for the purpose of limiting the invention.

It is to be understood that the singular forms “a”, “an” and “the” include plural referents unless the context clearly dictates otherwise.

It should be emphasized that the term “comprises/comprising” when used in this specification is taken to specify the presence of stated features, steps or components but does not preclude the presence or addition of one or more other features, integers, steps, components or groups thereof.

The present system incorporates a method for chart review and chart audit approach to enhance accuracy in risk adjustment calculations. It emphasizes the importance of accurate diagnosis coding, particularly through Hierarchical Condition Categories (HCCs), to accurately project future care costs. By implementing best practices in HCC coding, the system aims to mitigate financial risks, optimize beneficiary care, and address compliance requirements and audit challenges faced by health plans. Overall, the system provides an improved reimbursement approach that enhances accuracy, reduces financial risks, and ensures appropriate compensation aligned with the actual costs incurred by MA (Medicare Advantage) organizations within the Medicare Advantage program.

The data processing system and method for chart review and audit of natural language medical text for accurate risk adjustment provides a comprehensive solution to optimize chart review and chart audit workflows in risk adjustment. By leveraging NLP, ICD-10-CM code validation, MEAT knowledge, and a feedback loop, the system revolutionizes the accuracy, efficiency, and overall performance of risk adjustment processes, ultimately benefiting healthcare organizations and improving patient outcomes.

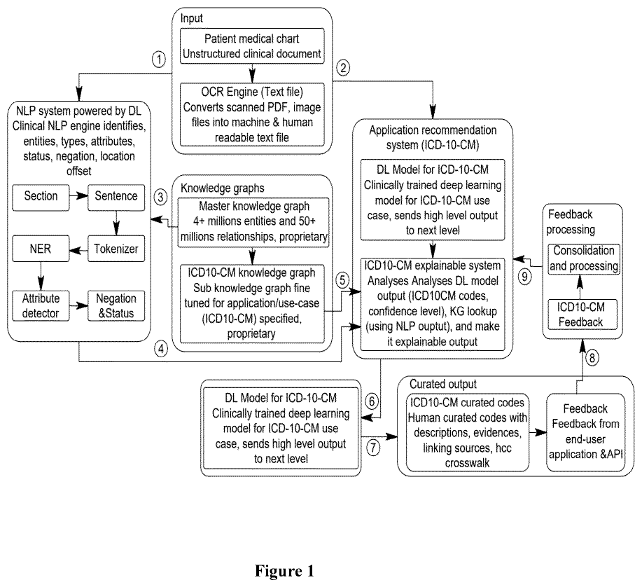

The FIG. 1 illustrates the method for optimizing chart review and audit workflows in accurate risk adjustment. Further, the present system leverages NLP, Machine Learning, knowledge graphs, and human expertise to provide accurate code recommendations and improve the overall coding process. Further, the system for operating the method for optimizing chart review and audit workflows in accurate risk adjustment, comprises an input document scanning module, an application recommendation module, an Natural Language Processing (NLP) module, an application module, an interface, and a feedback processing module.

Further, the input document scanning module is installed to scan one or more input documents in order to fetch one or more inputs. The said detailed level workflow is configured with the input document scanning module which takes documents as inputs wherein the said input documents includes documents such as medical charts in various formats (PDF files, scanned files, faxed files), as well as structured data in formats like HL7, FHIR, and CCDA, are fed into the NLP pipeline. In this stage, if the input file is in PDF format, an OCR (Optical Character Recognition) engine is employed to convert it into text format. Understanding the handwritten notes by the system is a difficult task. So, it can be verified by the human user. For that, this SaaS system's OCR module checks the input document if it has any handwritten notes, and if it finds any, then it will indicate to the user accordingly. So that the user could read and verify the handwritten notes.

Further, the Natural Language Processing (NLP) module receives inputs from primary knowledge graphs, which enrich the information by adding clinical context and establishing relations with other entities. Further, the Natural Language Processing (NLP) module adding the received data with one or more inputs and obtaining output. Further, the outputs are including but not limited to identification of entities, types, the attributes, status, navigation, location offset, explained in below mentioned example 1.

### Example 1

| NLP Capability | Description | Example |
|---|---|---|
| Document | Accurately classifying | Discharge summaries, |
| Classification | different types of | Radiology reports, etc. |
| | clinical documents | |
| Section Header | Identifying section | Past Medical History, |
| Identification | headers correctly | Family |
| | in clinical | History, Physical |
| | documents | Examination, etc. |
| Entity | Identifying | Diseases, symptoms, |
| and Relation | and categorize | medications, |
| Extraction | entities | procedures and |
| | | the relationships between |
| | | them, such as a medication |
| | | being used to treat a |
| | | particular disease. |
| Abbreviation | Correctly interpreting | “SOB” could mean |
| and Acronym | medical abbreviations | “shortness of breath” |
| Resolution | and acronyms | |
| | which could have | |
| | multiple meanings based | |
| | on the context | |
| Handling | Accurately detecting and | “Patient denies any history |
| Negation and | interpreting negations; | of diabetes” should not be |
| Assertion | Correctly identifying the | coded as an instance of |
| Status | status of a | diabetes. |
| | particular entity, | “Patient is experiencing |
| | such as whether | chest pain currently” is |
| | a condition is | asserting the patient's |
| | present, absent, | condition. |
| | possible, hypothetical, | |
| | or associated with | |
| | someone else like | |
| | family history. | |
| Temporal | Accurately | “Patient has a history of |
| Expression | recognizing and | seizures”. |
| Recognition | interpreting temporal | |
| | expressions, | |
| | distinguishing | |
| | between current and | |
| | historical | |
| | conditions or symptoms, | |
| | future planned | |
| | treatments, and so on. | |
| Experiencer | Identifying who the | “Patient's mother has a |
| Identification | experience of the | history of breast cancer”. |
| | condition is. | |
| Coreference | Capable of resolving | “The patient has diabetes. |
| Resolution | co-references | He takes insulin,” |
| | | the system should |
| | | understand that “He” |
| | | refers to “the patient.” |
| | | in this context. |
| Clinical Value | Capable of normalizing | Recognizing that “twice a |
| Normalization | clinical measurements and | day” and “BID” |
| | values extracted from the | refers to the same |
| | text. | medication frequency. |
| Semantics and | Integrating with standard | ICD-10-CM, HCC, |
| Ontology | medical terminologies and | SNOMED CT, RxNORM, |
| | ontologies and mapping the | etc. |
| | extracted entities to the | |
| | correct codes in these | |
| | ontologies. | |

The Natural language processing (NLP) is an interdisciplinary subfield of linguistics, computer science, and artificial intelligence concerned with the interactions between computers and human language, in particular how to program computers to process and analyze large amounts of natural language data. The goal is a computer capable of “understanding” the contents of documents, including the contextual nuances of the language within them. The technology can then accurately extract information and insights contained in the documents as well as categorize and organize the documents themselves.

The output are then passed through the application recommendation module, which is responsible for providing one or more recommendations on ICD-10-CM codes. In health care, diagnosis codes such as ICD-10-CM codes are used as a tool to group and identify diseases, disorders, symptoms, poisonings, adverse effects of drugs and chemicals, injuries and other reasons for patient encounters. Diagnostic coding is the translation of written descriptions of diseases, illnesses and injuries into codes from a particular classification. In medical classification, diagnosis codes are used as part of the clinical coding process alongside intervention codes. Both diagnosis and intervention codes are assigned by a health professional trained in medical classification such as a clinical coder or Health Information Manager.

The ICD-10-CM Code recommendation engine leverages a deep learning (DL) pipeline, where a comprehensive knowledge graph encompassing all 70,000 ICD-10-CM Codes is utilized. The system enables the mapping and prioritization of the most relevant and critical codes applicable to the patient's input document. The integration of deep learning techniques with the rich, interconnected data in the knowledge graph ensures accurate and contextually appropriate code recommendations.

The Application Recommendation module serves a vital role in ensuring transparency and explain ability within the AI module. Unlike a black-box system, this module provides a clear and comprehensive understanding of the AI system's decisions by offering supporting evidence and justifications for the suggested diagnosis (ICD-10-CM code). The application recommendation module of the present invention is a ML model for ICD-10-CM which is a clinically trained Machine Learning model for ICD-10-CM use case which sends high level output to the next level. The ML model output (ICD-10-CM codes, confidence level), KG lookup (using NLP output) and generates an explainable output.

However, the current reimbursement model presents challenges as monthly advance payments from the Centers for Medicare & Medicaid Services (CMS) to MA (Medicare Advantage) organizations are based solely on projected costs, leading to potential financial uncertainties. To address these challenges, this invention introduces an innovative workflow for the interpretation of medical charts of patients. The system leverages advanced technologies and streamlined processes to ensure accurate extraction of important data from the said medical charts of the patients to enable reimbursement aligned with the actual costs incurred by MA organizations, optimizing beneficiary care.

The present system implements an NLP powered by DL (Deep Learning). Deep learning is a machine learning technique that teaches computers to do what comes naturally to humans i.e. to learn by example. The NLP system of the present invention relates to a clinical NLP engine which identifies entities, types, the attributes, status, navigation, location offset etc. The Natural Language Processing (NLP) module adds the received data with the one or more recommendations to obtain the one or more output for identification of entities, types, the attributes, status, navigation, location offset etc.

The one or more output from the said NLP module is then forwarded to the application module. The said application module incorporates inputs from an application-specific Knowledge Graph (ICD-10-CM knowledge graph) to enhance the context of its findings. The application module provides one or more suggestions comprising ICD-10-CM codes, descriptions, diagnosis evidence, MEAT evidence, cross-walk to HCC codes, and RAF scores of HCC codes.

At least one user is involved in the coding process and performs various curation tasks. The said user reviews and validates the one or more suggestions, and the validation may involve accepting the suggestions as they are, making updates, rejecting suggestions, or adding new codes with supporting evidence from medical charts. The said updates may include editing codes, adding or removing evidence. The said human-in-the-loop may include a Coder, a reviewer, an auditor. There may be a plurality of user in the-loop.

During the said process of curation by the user in-the-loop, feedback is collected and sent to a feedback processing module. The said feedback processing module handles the collected feedback, making decisions on updating the application module based on consolidated feedback. This may involve adding new rules, fine-tuning models, and other specific actions.

Knowledge Graphs serve as intricate networks of entities (subjects, objects) and their relationships (properties), providing a structured and meaningful organization of information. These graphs can vary in size and shape, encompassing both human curated and automatically generated content, and can be either fixed or continuously expanding.

A knowledge graph, also known as a semantic network, represents a network of real world entities i.e. objects, events, situations, or concepts—and illustrates the relationship between them. This information is usually stored in a graph database and visualized as a graph structure, prompting the term knowledge “graph.” A knowledge graph is made up of three main components: nodes, edges, and labels. Any object, place, or person can be a node. An edge defines the relationship between the nodes.

A key principle of Knowledge Graphs involves explicitly describing relations among entities using SPO triples (Subject-Predicate-Object). This structured representation allows for comprehensive understanding. For example, a patient's medical chart may indicate that the patient is taking Atenolol, which is a subclass of Beta Blocker. The Knowledge Graph further reveals that Beta Blockers are medications used to treat high blood pressure. Consequently, through contextual inference, it can be determined that the patient is taking Beta Blocker medication for blood pressure control.

In the context of the present invention, the Clinical Knowledge Graphs comprise a primary knowledge graph (KG) and one or more application specific graphs. The primary knowledge graph represents the general knowledge graph with clinical entities and its relationships. The one or more application specific knowledge graphs are, including ICD-10-CM knowledge graph, MEAT knowledge graph used to identify a particular ICD-10-CM code by navigating through the clinical entities and its relationships, and to identify the MEAT evidence for a given ICD-10-CM code by navigating through the clinical entities respectively.

The Clinical Knowledge Graphs are meticulously curated using millions of diverse clinical charts, capturing millions of actual clinical entities and establishing numerous relationships. Continuously expanding, our Knowledge Graphs remain up to date by incorporating new knowledge and information.

The application recommendation system generates a suggested optimal output in the form of ICD-10-CM suggested codes which are system suggested codes with description, evidences, linking sources, confidence level, HCC crosswalk, etc. From the said output a curated output is generated. The said curated output is ICD-10-CM curated codes which are human curated codes with descriptions, evidences, linking sources, HCC crosswalk.

Feedback from the end-user application and API is sent for feedback processing where the said feedback is consolidated and processed and fed into the application recommendation system. Further, the interface is configured to collect a plurality of feedback, wherein the user is involved in development of a plurality of instructions and perform a plurality of curation tasks based on the one or more suggestions.

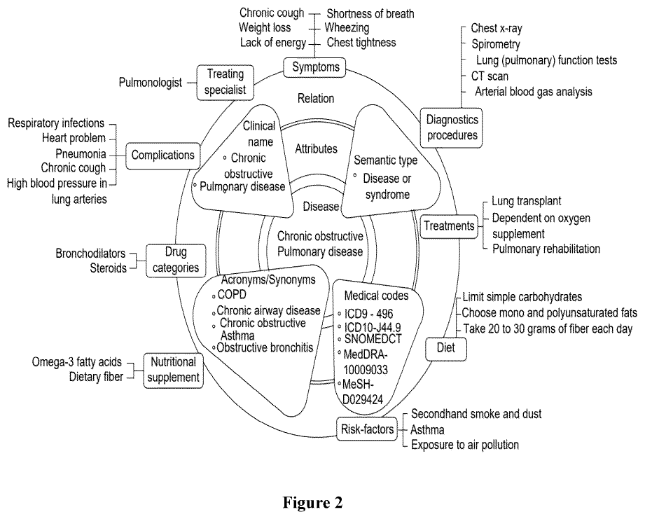

The FIG. 2 illustrates and demonstrates the traversal capabilities of a sample Knowledge Graph, allowing exploration from both the inside out and the outside in. This interactive navigation enhances the understanding of entity interconnections, making our Knowledge Graphs valuable tools for various applications.

A plurality of knowledge graphs are implemented for the present workflow. The said Knowledge graphs include the following:
Core Knowledge Graph: The said comprehensive knowledge graph serves as a foundational source of information, providing a broad understanding of relevant entities and their relationships. It forms the bedrock of knowledge for the entire workflow, facilitating accurate assessments and evaluations.
ICD-10-CM Knowledge Graph: The said knowledge graph is focused specifically on the ICD-10-CM coding system, this specialized graph encompasses a wealth of knowledge related to diagnostic codes. It assists in the accurate classification of medical conditions and supports precise documentation and reporting.
MEAT (Monitor, Evaluate, Assess, and Treat) Knowledge Graph: The MEAT knowledge graph is designed to optimize clinical decision-making and patient care, this dynamic graph integrates various data points and guidelines related to monitoring, evaluation, assessment, and treatment. It empowers healthcare professionals with evidence-based insights to drive informed decisions and promote effective care management.

By implementing the said diverse knowledge graphs, the Chart Review and Audit workflow benefits from enriched information and contextual understanding. This allows for more accurate reviews, audits, and subsequent actions, ultimately enhancing the quality of healthcare delivery and outcomes.

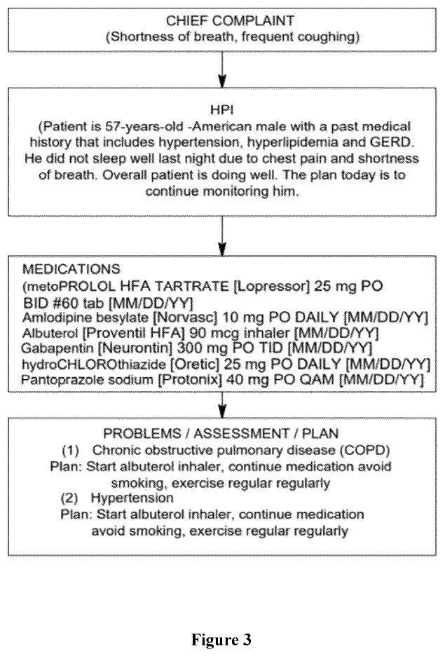

The FIG. 3 illustrates the unstructured data in a medical chart of a patient. An Electronic Health Record (EHR) is a digital version of a patient's medical chart, encompassing a comprehensive collection of the patient's medical history and care over time. It includes demographic details, medical history, medication lists, vital signs, lab results, imaging reports, treatment plans, and orders and prescriptions. The information is structured and mentioned into different sections like Chief complaint, History of present illness, Past medical/family/social history, Physical exam, Review of systems, Medications, Lab results, Assessment and plan, etc. EHRs enhance the accuracy, efficiency, and accessibility of patient information, contributing to improved healthcare outcomes.

Extracting accurate and contextually relevant information from medical records is essential. Various factors must be considered to ensure precision. For instance, the document is divided into multiple sections, each containing context-dependent information. Diseases listed in the family history section refer to the patient's family members, not the patient. Additionally, it's important to consider the assertions of entities, such as whether a condition is positive, negative, possibly positive, possibly negative, or uncertain, to fully understand the context. Similarly, the certainty and status of an entity-whether it pertains to the present, history, family history, or the future must also be considered to maintain the proper context.

Moreover, many complex medical terms have meanings that depend on the context. For example, the word “drainage” can refer to either a problem or a procedure, depending on its usage. The document also contains numerous abbreviations and templates for certain entities that require precise contextual extraction, which can be challenging. For instance, “m/r/g” stands for murmurs/rubs/gallops, which are signs and symptoms. Extracting such complex medical entities is crucial for fully understanding the medical record.

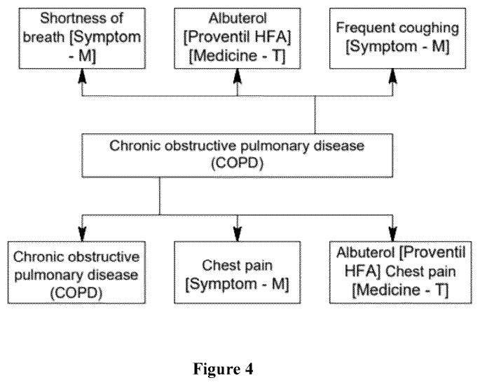

The FIG. 4 illustrates an example of a MEAT knowledge graph. In the said Example, the system receives the text extracted from an unstructured medical chart of FIG. 3 and performs a series of operations comprising:

Disease Identification: The system carefully examines the chart to identify any mentioned diseases or medical conditions, ensuring comprehensive analysis.

Knowledge Graph Utilization: Leveraging the power of Knowledge Graphs, the system retrieves disease-related details such as symptoms, medicines, laboratory data, instructions, and more. This deep integration with the Knowledge Graph enhances the system's understanding and accuracy.

Chart Validation: The system cross-references the extracted information from the chart against the derived list obtained from the Knowledge Graph, ensuring alignment and validation of the identified data.

Categorization with MEAT: Based on the extracted information, the system assigns the relevant details to their respective MEAT (Monitor, Evaluate, Assess, Treatment categories. This categorization enables streamlined organization and structured analysis of the chart's contents.

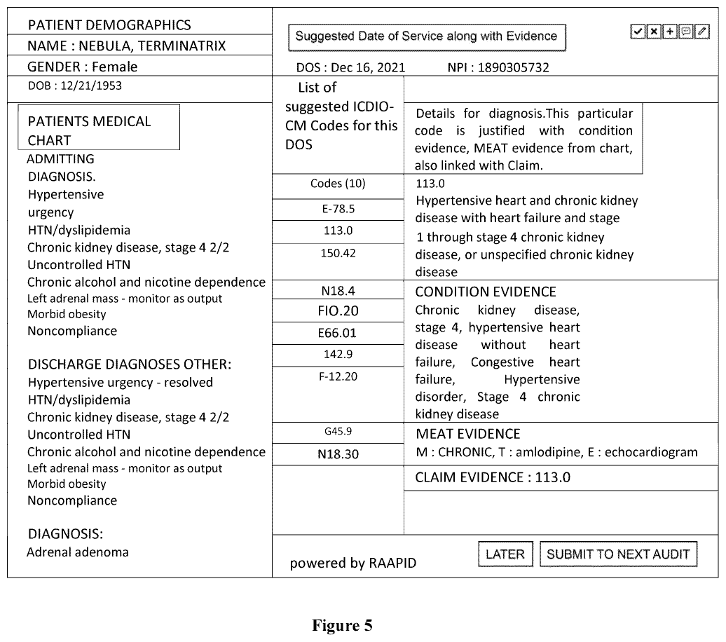

By implementing the said workflow, the system produces an output that presents the findings in a structured and meaningful manner, as shown in FIG. 5. This output serves as a valuable resource for healthcare professionals, facilitating efficient decision-making and ensuring comprehensive patient care. The system takes the unstructured medical chart and structured claim data as input. Then it process them by using NLP and Knowledge Graphs modules, and by using the Application Recommendation System, it identifies and suggests the Date of Service along with evidence; also identifies, suggests diagnosis codes (ICD-10-CM) for this DoS along with justifying condition evidence or diagnosis evidence from chart, MEAT evidence from chart, and claim evidence from claim. Optionally it could also find the patient demographic details as well.

Operating at the application layer of the workflow, this system seamlessly integrates various components to cater specifically to the use case of ICD-10-CM coding. It harmoniously combines multiple elements to deliver a cohesive and well-rounded recommendation process, enhancing the accuracy and reliability of the system's outputs.

By incorporating evidence-based reasoning and providing insightful justifications, the Application Recommendation System empowers healthcare professionals with valuable information and ensures a transparent and trustworthy AI-driven workflow.

For feedback processing, during the coding process, the coders engage with the suggested output in several ways, for each suggested code per Date of Service (DOS):

Accepting Suggestions: Coders accept the suggested ICD-10-CM code, description, Condition evidence, MEAT evidence, and Claim evidence without any modifications. This indicates that the system has performed impeccably, receiving a 100% endorsement from the coder. This applies to both the code and DoS recommendations.

Updating Suggestions: Coders have the option to update the suggested code by modifying it, adding or removing condition evidence, MEAT evidence, or adjusting the DoS. This indicates that while the system has performed reasonably well, there is room for improvement. This applies to both the code and DoS recommendations. Adding New Codes or DoS: Coders may introduce entirely new codes along with relevant evidence that the system missed entirely. They can also include previously unrecognized DoS. This indicates that the system's performance has been inadequate, as per the coder's assessment.

Ignoring Codes or DoS: Coders may choose to ignore the suggested condition or DoS, keeping them solely in the Suggested Codes section. This signifies that coders do not take any explicit action but may intend to reject the suggestion. This allows the coder to maintain the suggested information without further action.

Rejecting Codes or DoS: Coders explicitly reject codes or DoS if they believe the suggestions are incorrect. This communicates the coder's disagreement with the system's recommendations and indicates a desire for the system to learn from their explicit rejection.

By delineating the various ways coders interact with the suggested output, the system accommodates different preferences and feedback, ensuring a more robust and user centric coding experience.

Based on the interactions described above, the collection of feedback can be categorized into two distinct methods: implicit and explicit.

Implicit Feedback: Implicit feedback refers to feedback that is not directly expressed by the user. In this case, the user does not provide explicit feedback within or to the system. Instead, the system automatically collects feedback based on the type of interactions observed. Actions such as accepting, updating, adding, or ignoring suggestions are considered implicit feedback. The system captures these actions automatically without requiring the user to enter specific feedback. This applies to both the Software-as-a-Service (SaaS) user interface (UI) and API interactions. For SaaS, the UI layer and its middleware capture these actions and pass the necessary data to the Feedback Processing module. Similarly, for API, there should be an API available to collect these actions, either through response parameters or dedicated endpoints.

Explicit Feedback: Explicit feedback refers to feedback that is explicitly provided by the user within or to the system. In this case, the user directly expresses their feedback. The system collects this feedback based on the user's interactions, specifically when the user rejects a particular condition. When rejection occurs, the system prompts the user to specify the reason for rejection. This explicit feedback is applicable to both the SaaS UI and API interactions. For SaaS, the UI layer and its middleware capture the rejection action along with the reason, and pass the necessary data to the Feedback Processing module. Similarly, for API, there should be an API available to collect the rejection action along with the reason, either through response parameters or dedicated endpoints.

By distinguishing between implicit and explicit feedback, the system ensures comprehensive feedback collection, incorporating both user actions and user-stated feedback. This allows for a more thorough understanding of user preferences and contributes to system improvement in both the SaaS UI and API environments. Reasons for Rejection:

When a coder rejects a code, whether it is for a condition or a date of service, it is important to provide a reason for the rejection. Based on our understanding of coders' actions from past projects, general experience, and logical conclusions, the following reasons for rejecting codes have been identified. Additional reasons may be added as we continue to learn more in Risk Adjustment coding.

For Codes:
When rejecting a condition, coders may mention the following reasons:
Over claimed Condition: The condition has been claimed excessively.
Duplicate Condition: The same condition has already been mentioned elsewhere.
More Specific Code Available: A more specific code exists for the given condition.
Guideline/Rule Excludes: The guideline or rule specifically excludes the assigned code.
Wrong Code Mapping: The assigned code does not accurately reflect the condition.
Wrong Evidence: The supporting evidence does not align with the assigned code.
Wrong Section: The code belongs to an incorrect section or category.
No Proper MEAT Evidence: MEAT (Monitor, Evaluate, Assess, and Treat) evidence is lacking or insufficient.
Not Confident: The coder is not confident in the accuracy or appropriateness of the assigned code.
NLP Misinterpretation: The natural language processing (NLP) system has misinterpreted the information.
Repeated Mistake: The same mistake has been made repeatedly.
Others/Enter a Specific Reason: A specific reason for rejection can be entered.

For Date of Service:
When rejecting a date of service, coders may mention the following reasons:
Not a Valid Provider Signature: The provider signature is not valid or missing.
Not a Valid Provider Type: The assigned provider type is not valid for the given service.
Not a Valid Date of Service: The assigned date does not correspond to a valid service date.
Date of Service Out of Range: The assigned date is outside the acceptable range.
Guideline/Rule Excludes: The guideline or rule specifically excludes the assigned date.
Member Name Mismatch: The member's name does not match the assigned date of service.
Incorrect Member: The assigned member for the date of service is incorrect.
Missing Date of Birth: The date of birth for the member is missing or inaccurate.
Not a Valid Document: The document containing the date of service is not valid or reliable.
NLP Misinterpretation: The NLP system has misinterpreted the information related to the date of service.
Repeated Mistake: The same mistake has been made repeatedly.
Others/Enter a Specific Reason: A specific reason for rejection can be entered.

By providing clear and specific reasons for code rejection, the system can gather valuable feedback and improve accuracy in both condition and date of service assignments.

A method for optimizing chart review and audit workflows in accurate risk adjustment, comprising the following steps—

One or more inputs are received, including a structured input and an unstructured input, from a user, wherein the structured input having a number of formats and the unstructured input having one or more input documents, at step 1.

The one or more inputs are passed through an application recommendation module for providing one or more recommendations on diagnosis codes, wherein the diagnosis codes are used as a tool to group and identify diseases, disorders, symptoms, poisonings, adverse effects of drugs and chemicals, injuries and other reasons for patient encounters, at step 2.

The data is received, from a primary knowledge graph, by the Natural Language Processing (NLP) module and adding the received data with the one or more recommendation to obtain the one or more output, wherein the one or more output including but not limited to identification of entities, types, the attributes, status, navigation, location offset, at step 3.

The output is passed to an application module, where it uses the secondary knowledge graphs to find the application specific details, then it combines the findings to provide suggested output, at step 4.

The one or more suggestions scrutinized and validated by the user, wherein the user involved in development of a plurality of instructions and perform a plurality of curation tasks, at step 5.

A plurality of feedback is collected, during execution of the plurality of curation tasks, by the user, at step 6.

The plurality of feedback is transmitted to a feedback processing module for making decisions for updating the application module, based on the collected plurality of feedback, including addition of new rules, fine-tuning of models, at step 7.

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of the appended claims.

We claim:

1. A method for optimizing chart review and audit workflows in accurate risk adjustment, comprising:
(a) receiving one or more inputs, including a structured input and a unstructured input, from a user, wherein the structured input having a number of formats and the unstructured input having one or more input documents;
(b) receiving data, from a primary knowledge graph, by the Natural Language Processing (NLP) module and adding the received data with one or more inputs and obtaining output, wherein the outputs are including but not limited to identification of entities, types, the attributes, status, navigation, location offset;
(c) passing the output through an application recommendation module for providing one or more recommendations on diagnosis codes, wherein the diagnosis codes are used as a tool to group and identify diseases, disorders, symptoms, poisonings, adverse effects of drugs and chemicals, injuries and other reasons for patient encounters;
(d) passing the one or more output to an application module, wherein the application module receives one or more information from the application specific one or more secondary knowledge graphs, and combine with the one or more outputs in order to provide one or more suggestions;
(e) scrutinizing and validating the one or more suggestions by the user, wherein the user involved in development of a plurality of instructions and perform a plurality of curation tasks;
(f) collecting a plurality of feedback, during execution of the plurality of curation tasks, by the user; and
(g) transmitting the plurality of feedback to a feedback processing module for making decisions for updating the application module, based on the collected plurality of feedback, including addition of new rules, fine-tuning of models.

2. The method as claimed in claim 1, wherein said one or more input documents include but not limited to medical charts.

3. The method as claimed in claim 1, wherein said diagnosis codes include but not limited to ICD-10-CM codes.

4. The method as claimed in claim 1, wherein the one or more suggestions include but not limited to ICD-10-CM codes, descriptions, diagnosis evidence, MEAT evidence, cross-walk to HCC codes, and RAF scores of HCC codes

5. The method as claimed in claim 1, wherein said number of formats include but not limited to PDF files, scanned files, faxed files, HL7, FHIR, and CCDA.

6. The method as claimed in claim 1, wherein said one or more recommendations include but not limited to identify diseases, disorders, symptoms, poisonings, adverse effects of drugs and chemicals, injuries.

7. The method as claimed in claim 1, wherein said one or more suggestions include but not limited to ICD-10-CM codes, descriptions, diagnosis evidence, MEAT evidence, cross-walk to HCC codes, and RAF scores of HCC codes.

8. The method as claimed in claim 1, wherein the process of scrutinizing and the validating of the one or more suggestions include but not limited to accepting and rejecting the one or more suggestions, making updates and adding new instructions with supporting evidence from said medical charts.

9. The method as claimed in claim 1, wherein the primary knowledge graph includes but not limited to core knowledge graph.

10. The method as claimed in claim 1, wherein the secondary knowledge graph includes but not limited to ICD10-CM knowledge graph, MEAT knowledge graph.

11. The method as claimed in claim 1, wherein the plurality of feedback, including:
an implicit feedback, based on the curation tasks including accepting, updating, and adding of actions, automatically collects the feedback; and
an explicit feedback, based on the curation task including deletion of an action, requires confirmation from the coder.

12. The method as claimed in claim 1, wherein the knowledge graph including:
i. a core knowledge graph having a source of information and facilitating assessments and evaluation;
ii. a ICD-10-CM knowledge graph facilitating classification of medical conditions and supports precise documentation and reporting; and
iii. MEAT (Monitor, Evaluate, Assess, and Treat) Knowledge Graph facilitating evidence-based insights to drive informed decisions and promote effective care management.

13. The system for operating the method for optimizing chart review and audit workflows in accurate risk adjustment, comprises:
I. an input document scanning module installed to scan the input document in order to extract one or more input, wherein the input document in PDF format converted into text format by an Optical Character Recognition (OCR);
II. an Natural Language Processing (NLP) module installed to fetch data from the primary knowledge graph and adding the received data with the one or more input to obtain the one or more output;
III. an application recommendation module installed for providing recommendations on ICD-10-CM codes based on the one or more output;
IV. an application module installed to receives one or more information from application specific one or more secondary knowledge graphs and combine with the recommendations in order to provide one or more suggestions;
V. an interface to collect a plurality of feedback, wherein the user involved in development of a plurality of instructions and perform a plurality of curation tasks based on the one or more suggestions; and
VI. a feedback processing module for making decisions for updating the application module, based on the collected plurality of feedback.

14. The system as claimed in claim 11, wherein the Natural Language Processing (NLP) module implements deep learning and machine learning techniques to obtain the one or more output.
